(19) United States
(12) Patent Application Publication
(10) Pub. No.: US 2006/0200028 A1
(43) Pub. Date: Sep. 7, 2006

(54) Sensor-based apparatus and method for portable noninvasive monitoring of blood pressure

(75) Inventor: Kevin Ray Evans, New Richmond, WI (US)

Correspondence Address: ALTERA LAW GROUP, LLC, 6500 CITY WEST PARKWAY, SUITE 100, MINNEAPOLIS, MN, 55344-7704, US

(73) Assignee: Medwave, Inc.

(21) Appl. No.: 11/072,916

(22) Filed: Mar. 4, 2005

(51) Int. Cl. A61B 5/02

(52) U.S. Cl. 600/485

(57) ABSTRACT

Blood pressure including systolic pressure, diastolic pressure, and pulse rate may be determined from a portable monitoring device that noninvasively senses at the surface of a patient's body pressure pulses that are influenced by blood flow in an underlying artery. As varying hold-down pressure is applied to the artery through overlying tissue, the pressure pulses are sensed by a transducer to produce waveform data. The varying pressure is applied automatically in a predetermined pattern using a pneumatic system, and is preferably swept in an increasing fashion so the waveform data from a series of pressure pulses are obtained with different amounts of force being applied. The waveform data from the sensed pressure pulses is analyzed to determine waveform parameters, and blood pressure is calculated in the portable monitoring device based upon the waveform parameters.

### BACKGROUND OF THE INVENTION

1. Field of the Invention

The present invention relates to monitoring of arterial blood pressure, and more particularly to the portable noninvasive monitoring of arterial blood pressure, including systolic pressure, diastolic pressure, mean pressure, pulse rate, and pressure waveform characteristics.

2. Description of the Related Art

Various different methods may be used to measure blood pressure: invasive, oscillometric, auscultatory, tonometric, and sensor-based. The invasive method, which is known as an arterial line (A-Line), involves insertion of a needle into the artery and is generally accepted as the “gold standard.” The other methods are noninvasive. The oscillometric method determines blood pressure from the amplitude of pressure oscillations in a pressurized cuff, typically measured within the cuff while the cuff is slowly deflated. The auscultatory method involves monitoring Korotkoff sounds as an inflated cuff placed around a cooperating artery of the patient slowly deflates. Systolic pressure is indicated when Korotkoff sounds begin to occur, while diastolic pressure is indicated when the Korotkoff sounds become muffled or disappear. The tonometric method typically uses an array of pressure sensitive elements which have at least one dimension smaller than the lumen of the underlying artery in which blood pressure is to be measured. The array is pressed against the site to measure a reference pressure directly from the wrist, which is correlated with arterial pressure.

The oscillometric, ausculatory, and tonometric methods have not been entirely satisfactory. Because both the oscillometric and the auscultatory methods require inflation of a cuff, they are not entirely suitable for performing frequent measurements and measurements over long periods of time. The frequency of measurement is limited by the time required to inflate and deflate the cuff, and the pressure imposed by the cuff is uncomfortable to the patient. Moreover, both the oscillometric and auscultatory methods lack accuracy and consistency. While the tonometric method eliminates the need for a cuff, accurately positioning and maintaining the individual pressure sensitive elements over the underlying artery is difficult. The tonometric method requires that the system be calibrated to compensate for gain, which is the ratio of pressure outside the artery to the pressure inside the artery. Improper placement will make calibration ineffective, and patient movement during measurement will change the gain and affect the accuracy of the measurement.

Various noninvasive sensor-based approaches have overcome the disadvantages of the invasive, oscillometric, auscultatory and tonometric methods. One noninvasive sensor-based approach is the wrist mounted blood pressure sensor device described in U.S. Pat. No. 5,640,964 issued Jun. 24, 1997 to Archibald et al. The '964 patent describes a device for supporting a sensing surface above an underlying artery of a patient. The device includes a hold down assembly and a sensor interface pivotally coupled to the hold down assembly. The sensor interface includes a mount, a compressible side wall extending from the mount, and a flexible diaphragm secured at the bottom of the side wall. The flexible diaphragm has an active portion for transmitting blood pressure pulses of the underlying artery, and the compressible side wall encircles the active portion. The mount has a connection located below the top of the compressible side wall, and a movable member extends between the hold down assembly and the connection so that the movable member is pivotally coupled to the sensor below the top of the compressible side wall.

Another noninvasive sensor-based approach is the wrist mounted blood pressure sensor device described in U.S. Pat. No. 6,558,335 issued May 6, 2003, to Thede. The '335 patent describes a device that includes a housing having a sensing region and a pivot region. The sensing region is pivotable about the pivot region in response to a hold down pressure applied at the sensing region by a user. The device includes a sensor interface assembly that is supported by the sensing region. The sensor interface assembly includes a sensing surface suited for engaging tissue adjacent the artery for sensing pressure from the artery. A wrist connection holds the housing adjacent the patient's wrist.

While both approaches described in the '964 patent and in the '335 patent have been successful, even greater convenience in the noninvasive sensing of blood pressure without sacrificing accuracy is desired.

### BRIEF SUMMARY OF THE INVENTION

The present invention provides a noninvasive sensor-based approach to detecting and measuring arterial blood pressure. The approach is particularly well suited to portability, and is convenient for the user without sacrificing accuracy.

These and other advantages are realized in varying degrees by the various embodiments of the present invention. One embodiment of the present invention is an apparatus for portably and non-invasively monitoring blood pressure of a patient, comprising a body having a first attachment site spaced apart from a second attachment site over an intervening region of the body; a pneumatically actuated pressure applicator mounted to the body; a sensor; a pneumatic pump mounted to the body and pneumatically coupled to the pressure applicator; and a control system mounted to the body, the control system being electrically coupled to the pressure transducer and electrically coupled to the pump. The sensor comprises a support member movably coupled to the pressure applicator, and extendable and retractable relative to the intervening body region by the pressure applicator; a pressure transducer; and a pressure pulse transmission medium having a sensing surface for contacting tissue of the patient. The pressure transmission medium is supported by the support member and coupled to the pressure transducer for conveying pressure pulses thereto from the sensing surface.

Another embodiment of the present invention is an apparatus for portably and non-invasively monitoring blood pressure, comprising a rigid casing having a fulcrum site; a first anchor coupled to a first site on the casing; a second anchor coupled to a second site on the casing, an intervening portion of the casing between the first and second sites forming a lever and the fulcrum site being on a first side of the lever; a band having one end secured to the first anchor, and another end for being secured to the second anchor; a housing contained within the casing, the housing forming a first part of an air chamber; a rolling diaphragm having a truncated conical form when in an extend position, a large diameter end of the diaphragm being open and coupled to the housing, and a small diameter end of the diaphragm being closed for forming a second part of the air chamber, the rolling diaphragm being biased toward a collapsed position, and the air chamber having an increased volume with the rolling diaphragm in the extended position, and a decreased volume with the rolling diaphragm in the collapsed position; a piston affixed to the small diameter end of the diaphragm; a sensor post connected to the piston; a guide rod connected to the housing, the sensor post being in slidable engagement with the guide rod; a unitary pressure sensor; a pneumatic pump contained within the casing; an airflow restrictor contained within the casing, the pneumatic pump being pneumatically coupled to the airflow restrictor and the airflow restrictor being pneumatically coupled to the air chamber; a normally open pressure release valve pneumatically coupled to the air chamber; a control system contained within the casing and having a user interface accessible to a user from without the casing, the control system being electrically coupled to the pressure transducer, electrically coupled to the pump, and electrically coupled to the normally open pressure release valve for closing the valve during operation of the pneumatic pump; a battery contained within the casing and electrically coupled to the control system; and a positioning guide coupled to the casing in proximity to the fulcrum site. The positioning guide has an arc-like shape generally conformal with a cross-section of a human wrist, extends toward the sensor from the fulcrum site, has a hole through which the sensor passes, and has a positioning notch for receiving a finger to detect a distal end of a radius bone when the positioning guide in engaged with a patient's wrist. The second anchor is elongated and extends from the casing and past the positioning guide in a direction generally tangential thereto, and has a hole therein for accessing the positioning notch with the finger. The unitary pressure sensor comprises a sensor support member; a flexible ring extending from the sensor support member; a compressible ring extending from the flexible ring, a sensor interior being bounded by the sensor support member, the flexible ring, and the compressible ring; a pressure pulse transmission medium contained generally within the sensor interior; and a pressure transducer mounted within the sensor interior for receiving pressure pulses through the pressure pulse transmission medium. The sensor support member has a sensor mount recessed within the flexible ring and pivotally connected to the sensor post, the sensor being disposed away from the casing with the rolling diaphragm in the extended position, and disposed near to the casing with the rolling diaphragm in the collapsed position.

Another embodiment of the present invention is an apparatus for portably and non-invasively monitoring blood pressure of a patient, comprising a body comprising a control system; means for attaching the body to a monitoring site on an anatomical structure of the patient from which noninvasive monitoring of blood pressure may be performed; means for pneumatically extending a sensor against the monitoring site from the body with a varying hold-down pressure, under control of the control system; means for obtaining pressure data from the sensor, under control of the control system; means for calculating blood pressure from the pressure data, under control of the control system; and means for pneumatically releasing the hold-down pressure from the sensor to retract the sensor, under control of the control system.

Another embodiment of the present invention is a method for portably and non-invasively monitoring blood pressure of a patient, comprising attaching a body to a monitoring site on an anatomical structure of the patient from which noninvasive monitoring of blood pressure may be performed; pneumatically extending a sensor against the monitoring site from the body with a varying hold-down pressure, under control of a control system disposed in the body; obtaining pressure data from the sensor, under control of the control system; calculating blood pressure from the pressure data, under control of the control system; and pneumatically releasing the hold-down pressure from the sensor to retract the sensor. The hold-down pressure may be released under control of the control system, or automatically upon failure of the control system.

### BRIEF DESCRIPTION OF THE SEVERAL VIEWS OF THE DRAWINGS

FIG. 1 is a perspective top view of a sensor-based apparatus for portable noninvasive monitoring of blood pressure, as mounted on the wrist of a patient.

FIG. 2 is a perspective edge view of the sensor-based device of FIG. 1.

FIG. 3 is a side view of the sensor-based device of FIG. 1.

FIG. 4A is a top view of a base section of the sensor of the apparatus for portable noninvasive monitoring of blood pressure of FIGS. 1-3.

FIG. 4B is a sectional view of the base section of FIG. 4A.

FIG. 4C is a bottom view of the base section of FIG. 4A.

FIG. 5A is a top view of a sensing section of the sensor of the apparatus for portable noninvasive monitoring of blood pressure of FIGS. 1-3.

FIG. 5B is a sectional view of the sensing section of FIG. 5A.

FIG. 5C is a bottom view of the sensing section of FIG. 5A.

FIG. 6 is a top exploded view of the base section and the sensing section of the sensor of the apparatus for portable noninvasive monitoring of blood pressure of FIGS. 1-3.

FIG. 7 is a bottom exploded view of the base section and the sensing section of the sensor of the apparatus for portable noninvasive monitoring of blood pressure of FIGS. 1-3.

FIG. 8 is a cross-sectional view of the apparatus for portable noninvasive monitoring of blood pressure of FIGS. 1-3.

FIG. 9 is a perspective view of a rolling diaphragm and related components suitable for use in the apparatus for portable noninvasive monitoring of blood pressure of FIG. 8.

FIG. 10 is a block schematic representation of a pneumatic system suitable for use in the apparatus for portable noninvasive monitoring of blood pressure of FIG. 8.

### DETAILED DESCRIPTION OF THE INVENTION, INCLUDING THE BEST MODE

Blood pressure including systolic pressure, diastolic pressure, and pulse rate may be determined from a portable monitoring device that noninvasively senses at the surface of a patient's body pressure pulses that are influenced by blood flow in an underlying artery. As varying hold-down pressure is applied to the artery through overlying tissue, the pressure pulses are sensed by a transducer to produce waveform data. The varying pressure is applied automatically in a predetermined pattern using a pneumatic system, and is preferably swept in an increasing fashion so the waveform data from a series of pressure pulses are obtained with different amounts of force being applied. The waveform data from the sensed pressure pulses is analyzed to determine waveform parameters, and blood pressure is calculated in the portable monitoring device based upon the waveform parameters.

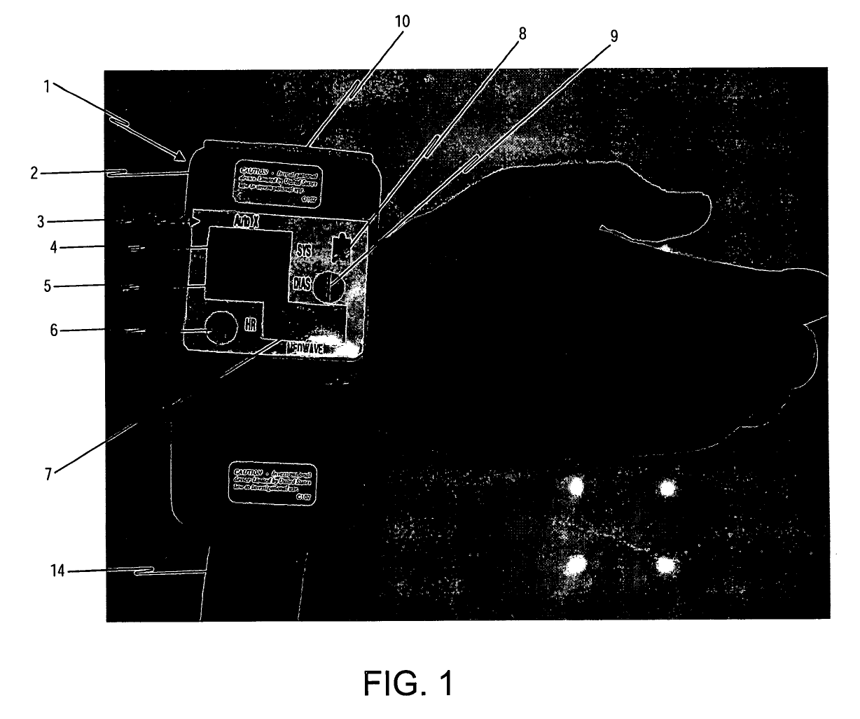

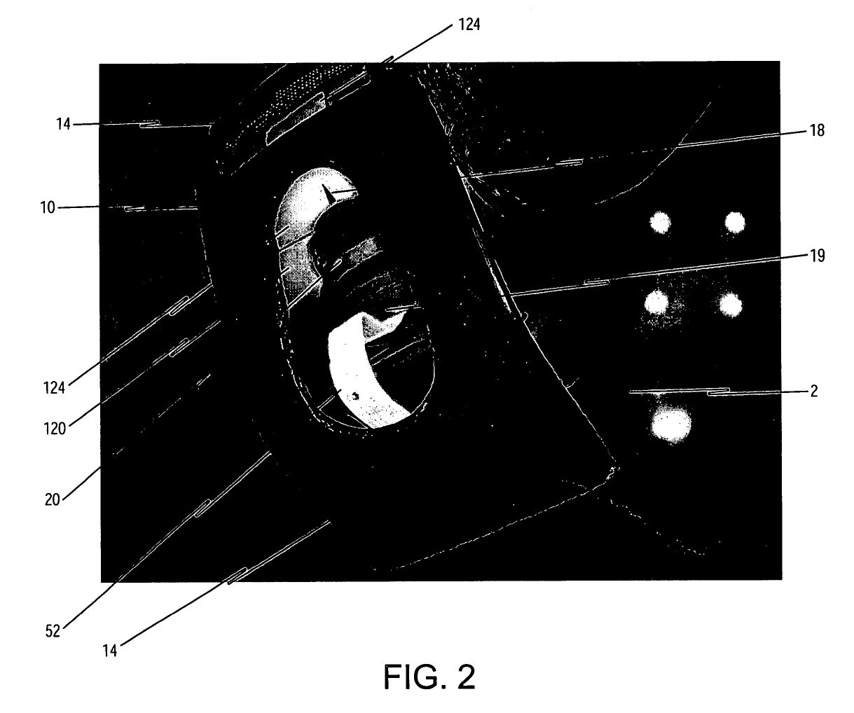

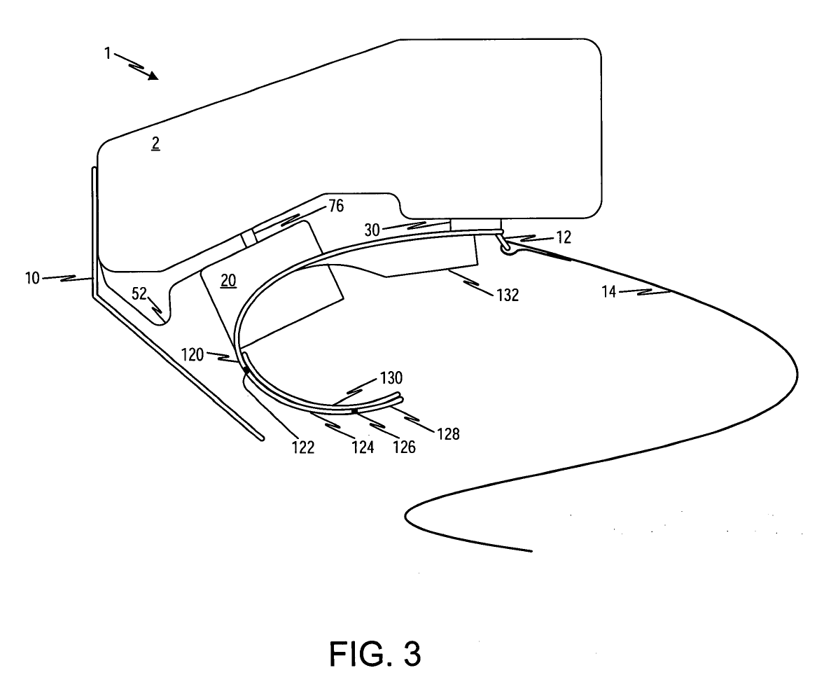

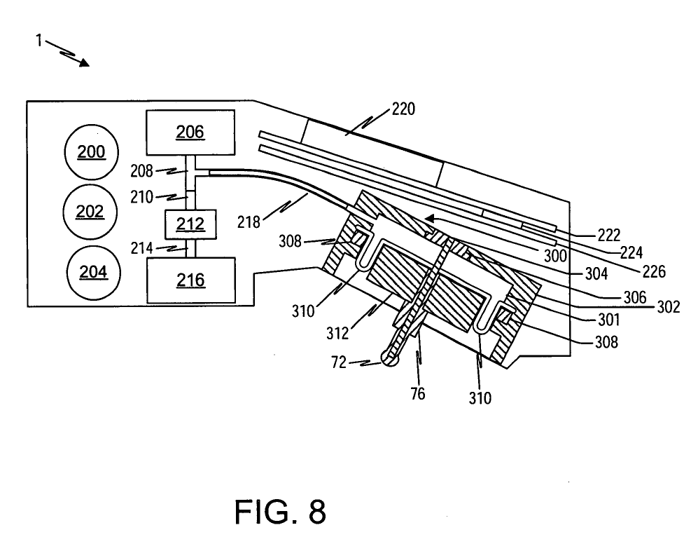

FIG. 1 and FIG. 2 are views from different perspectives, and FIG. 3 is a side view of a sensor-based device 1 for the portable noninvasive monitoring of blood pressure, as mounted on the wrist of a patient. The monitoring device 1 has a housing 2, a hold-down pressure generating unit 300 (FIG. 8) preferably mounted and contained within the housing 2, and a sensor 20 pivotally coupled to the hold-down pressure generating unit 300. The hold-down pressure generating unit 300 includes a control circuit, a pneumatic system, and a power source. A user interface panel 3 is electrically coupled to the control circuit, and includes a numeric indicator 4 for displaying systolic pressure, a numeric indicator 5 for displaying diastolic pressure, and a numeric indicator 7 for displaying pulse or heart rate. The user interface panel 3 also includes a master on/off switch 9, a hold-down pressure generator start/stop switch 6, and a battery indicator 8. The start/stop switch 6 is pressed to initiate a monitoring cycle in which a varying force is applied to the radial artery by the hold-down pressure generating unit 300, and the counter pressure in the radial artery produces a signal that is digitized and used to calculate the desired blood pressure. A pressurization cycle may be stopped anytime by pressing the start/stop switch 6 during the cycle. While the user interface panel 3 may be furnished with a variety of other indicators and controls, such as controls for selecting particular pulse waveforms for display and study on an LED screen (not shown), for example, the particular set of controls and indicators shown in FIG. 1 is particularly effective for rapidly and conveniently presenting the most useful information about blood pressure to the user. Other possible controls and indicators include a waveform trend display, table trend display, real time display, and controls for such functions as patient identification, cycle time, sensor height (height difference between the wrist sensor and heart level), alarm setup, and clock controls.

The monitoring device 1 is secured to the patient in any convenient manner, illustratively by strapping it on with a Velcro® brand strap 14 (FIG. 2 and FIG. 3). The ends of the strap 14 are looped through respective anchors 10 and 12, which are attached at or near opposite ends of the monitoring device 1. The anchor 12 is illustratively a U-shaped metal bracket rotatably mounted into a block 30 that projects from the casing 2. The anchor 10 is an elongated strip of flexible material that tends to maintain its shape, such as preformed plastic, which is secured to one end of the casing 2 by any suitable technique such as with glue or mechanical fasteners, and which extends away from the casing 2 either to hold the strap 14 away from the patient's arm which slipping the monitoring device 1 onto the patients arm, or to facilitate insertion of the strap 14 through a transverse slot at the distal end of the anchor 10. To locate the proper position for placement of the sensor 20, the user first palpates the arm with a finger to find the distal edge of the radius bone. The sensor 20 is then placed directly over this point, and the strap 14 is secured snugly. A preferably articulated placement guide that includes segments 120, 124 and 128 and articulation regions formed by regions of layer 130 under intervening gaps 122 and 126 helps in the proper placement. The placement guide is attached at one end of the segment 120 to the casing 2 by a mounting block 30. When the monitoring device 1 is applied to the patient, segments 120, 124 and 128 of the placement guide straddle the styloid process bone of the patient and generally guide the sensor 20 into position over the underlying artery and the radius bone. Indicator symbols such as notch symbol 18 (FIG. 2) on the placement guide segment 124 and arrow symbol 19 (FIG. 2) on the sensor 20 align to the distal edge of the radius bone when the sensor 20 is properly positioned, and proper placement may be verified tactilely by passing a finger through an elongated access slot in the anchor 10 and an access notch in the placement guide segments 120 and 124, and feeling the distal edge of the radius bone. The access notch extends from a aperture through which the sensor 20 passes. The sensor aperture may be any shape that does not impede movement of the sensor 20, the generally circular shape shown in FIG. 2 being illustrative. The portions of segment 120 that flank the aperture and access notch may be thought of as guide ribs which meet within segment 124. A suitable articulated placement guide is described in further detail in a copending U.S. patent application filed on even date herewith in the name of inventor Kevin R. Evans and entitled “Articulated placement guide for sensor-based noninvasive blood pressure monitor” (Attorney Docket No. 01845.0043-US-01), which hereby is incorporated herein in its entirety by reference thereto.

The use of the notch indicator symbol 18 and the arrowhead indicator symbol 19 is somewhat arbitrary, and other shapes conveying a sense of direction could be used as well. Good placement of the sensor over the radius bone causes both indicator symbols 18 and 19 to point to the distal edge of the radius bone. Poor placement causes neither of the indicator symbols 18 and 19 to point to the distal edge of the radius bone.

With the monitoring device 1 properly positioned, the monitoring device 1 is switched on by pressing the on/off switch 9, and a cycle is initiated by pressing the start/stop switch 6. As the hold-down pressure generating unit 300 operates, it moves the sensor 20 away from the casing 2 by extending a sensor post 76. The sensor 20 gently exerts pressure against the patient's wrist over the radial artery, while cushion 132 on the placement guide segment 120 and layer 130 extending across whole or parts of placement guide segments 120, 124 and 128 and spanning the intervening gaps 122 and 126 gently distribute pressure over other areas of the patient's wrist. The layer 130 illustratively is a strip of shaped flexible material that tends to return to its original shape after being flexed, while the segments 120, 124 and 128 are less flexible that the layer 130. The cushion 132 also functions as a pivot point about which the hold-down pressure is applied.

Since the sensor 20 is relatively small compared to the larger cuffs used with oscillometric and auscultatory methods, the sensor 20 applies a hold down pressure to only a relatively small area above the underlying artery of the patient. Consequently, blood pressure measurements may be taken with less discomfort to the patient. Because the sensor 20 does not require inflation or deflation, faster and more frequent measurements may be taken. Furthermore, the sensor 20 better conforms to the anatomy of the patient so as to be more comfortable to the patient, and the automatic application of the hold-down pressure avoids ineffective hold-down cycles and achieves consistent and accurate blood pressure measurements.

The device 1 may include an external connector (not shown) for transmitting and receiving data, recharging batteries contained within the casing 2, and provide an alternative power source to the device 1.

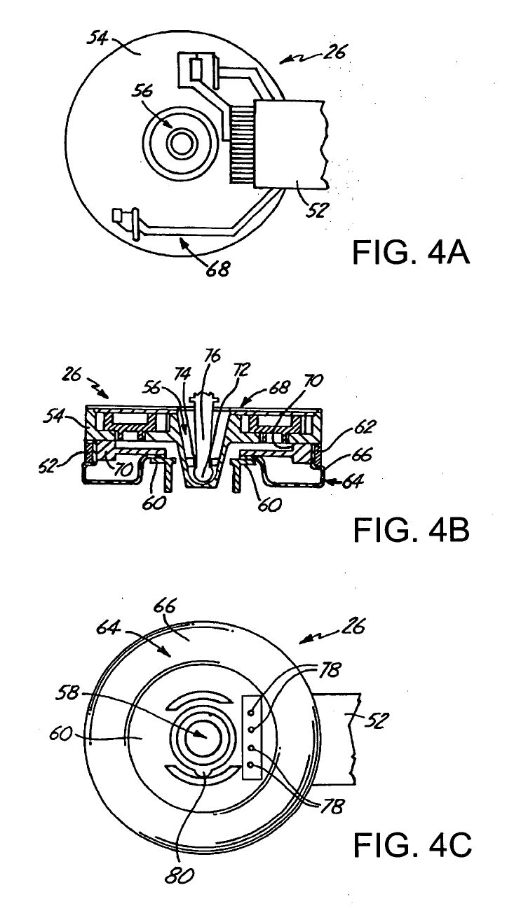

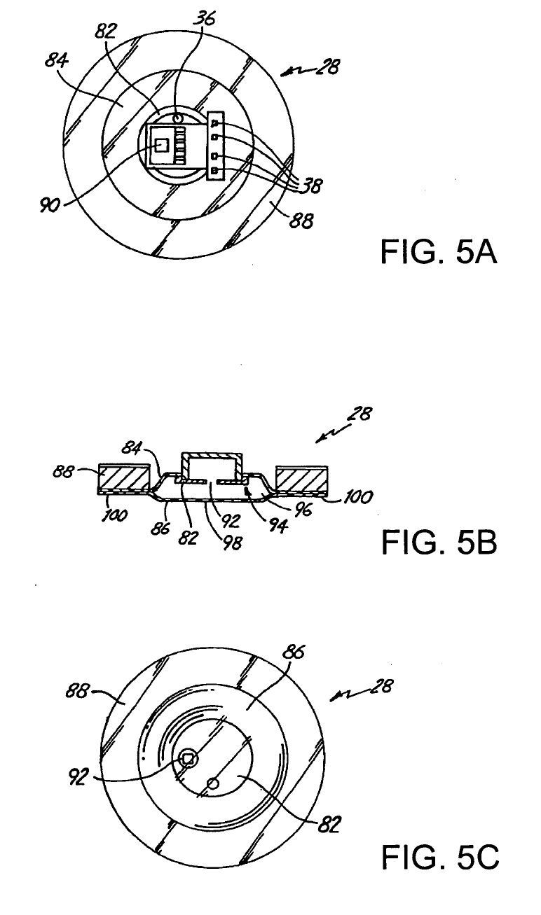

The sensor 20 is pivotally attached to the hold-down pressure generating unit 300 (FIG. 8) within the casing 2 by sensor post 76. While any pivoting joint may be used, illustratively the end of the sensor post 76 is provided with a ball 72 (FIG. 8) which fits into a corresponding socket 74 on a base section 26 (FIG. 4) of the sensor 20, and allows the sensor 20 to pivot near the wrist surface to accommodate the anatomy of the patient. Because the base section 26 is pivotal about a low pivot point, the sensor 20 is permitted to be positioned with stability above the underlying artery. In addition, the low pivot point enables the user to apply a more direct, uniform force on a sensing section 28 (FIG. 5). Thus, the hold down pressure is more uniformly applied to the anatomy above the underlying artery. As pressure is applied by the hold-down pressure generating unit 300, that force is transferred through the sensor post 76 and through the sensor 20.

FIGS. 4A-4C show top, sectional, and bottom views, respectively, of base section 26 of blood pressure measurement device 10. Base section 26 includes an electrical connector 52, illustratively a ribbon cable, a top plate 54, an upper receptacle 56, a lower receptacle 58, an inner mounting ring 60, an outer mounting ring 62, a flexible ring 64 that includes a side wall diaphragm 66, electrical circuitry 68, and an upper capture 70.

Electrical connector 52 electrically couples the base section 26 with electrical components within the casing 2. Additionally, power for sensing section 28 is delivered via electrical connector 52.

The base section 26 is pivotally joined to the hold-down pressure generating unit 300 by sensor post 76. The ball 72 is located at a lower end of the sensor post 76, and socket 74 is formed within a lower portion of upper receptacle 56 of the base section 26. The ball 72 is pivotally mounted in socket 74.

Sensing section 28 may be permanently attached to base section 26, or may be detachably joined to base section 26 by a mechanical connector 34 (FIG. 5A). Electrical connectors 78 and an alignment receptacle 80 may be located in the base section 26 for receiving the connector 34, illustratively in inner mounting ring 60 of lower receptacle 58.

Flexible ring 64 is defined by side wall diaphragm 66 and upper capture 70. Side wall diaphragm 66 is formed from a generally circular sheet of flexible material, such as polyurethane, and is preferably filled with fluid. Diaphragm 66 bulges outward when flexible ring 64 is filled with fluid. The outer edge portion of diaphragm 66 is held between top plate 54, outer ring 62 and upper capture 70. The inner edge portion of diaphragm 66 is held between inner ring 60 and upper capture 70. Ring 64 is compressible and expandable in the vertical direction so as to be able to conform to the anatomy of the patient surrounding the underlying artery. As a result, the distance between top plate 54 and the patient's anatomy can vary around the periphery of flexible ring 64 according to the contour of the patient's anatomy. Furthermore, because fluid is permitted to flow through and around ring 64, pressure is equalized around the patient's anatomy.

FIGS. 5A-5C show top view, sectional and bottom views, respectively, of sensing section 28 of the sensor 20. Sensing section 28 includes a diaphragm capture 82, an inner diaphragm 84, a flexible (or outer) diaphragm 86, a compressible ring 88, a pressure transducer 90 having a sensing surface 92, and connector 34. Inner diaphragm 84 and flexible diaphragm 86 form a sensor chamber 94 which is filled with a fluid coupling medium 96. Any of a variety of different types of pressure transducers may be used for the transducer 90, one suitable type being part number MPX2300DT1 or MPX2301DT1, which is available from Freescale Semiconductor, Inc. of Austin, Tex.

The connector 34 illustratively includes an alignment element 36 and electrical connectors 38. Electrical connectors 38 are connected to and extend from pressure transducer 90. Electrical connectors 38 mate with electrical connectors 78 located on the base section 26. Electrical connectors 38 provide the connection between transducer 90 and the electrical circuitry of the base section 26. Alignment element 36 is received by alignment receptacle 80 (not shown) of base section 26 to precisely position electrical connectors 38 within the corresponding electrical connectors 78 of base section 26. In one arrangement, the sensing section 28 may be individually detached from base section 26 and replaced by another sensing section. It will be appreciated that any suitable mating electrical connectors may be used for the electrical connectors 38 and 78; illustratively, electrical connectors 38 are receptacles or sockets, while electrical connectors 38 are recessed pins.

Compressible ring 88 is generally annular and may be formed from a polyurethane foam or other pulse dampening material, including open cell foam and closed cell foam. Ring 88 is centered about flexible diaphragm 86 and positioned above diaphragms 84 and 86. Compressible ring 88 is isolated from fluid coupling medium 96 within sensor chamber 94 formed by diaphragms 84 and 86. The compressibility of ring 88 allows ring 88 to absorb and dampen forces in a direction parallel to the underlying artery. The forces are exerted by the blood pressure pulses on sensing section 28 as the blood pressure pulses cross flexible diaphragm 86. Because compressible ring 88 is isolated from fluid coupling medium 96, the forces absorbed or received by ring 88 cannot be transmitted to fluid coupling medium 96. Instead, these forces are transmitted across compressible ring 88 and flexible ring 64 to top plate 54 (shown in FIG. 4B), which is a path distinct and separate from fluid coupling medium 96.

Rings 64 and 88 apply force to the anatomy of the patient to neutralize the forces exerted by tissue surrounding the underlying artery. Rings 64 and 88 are compressible in height, thus the height of the side of the sensor 20 decreases as the sensor 20 is pressed against the patient's wrist.

Inner diaphragm 84 is an annular sheet of flexible material having an inner diameter sized to fit around diaphragm capture 82. An inner portion of inner diaphragm 84 is trapped or captured, and may be adhesively affixed to the lip of diaphragm capture 82. Inner diaphragm 84 is permitted to initially move upward as flexible diaphragm 86 conforms to the anatomy of the patient surrounding the underlying artery. As compressible ring 88 is pressed against the anatomy of the patient surrounding the artery to neutralize or offset forces exerted by the tissue, flexible diaphragm 86 is also pressed against the anatomy and the artery. However, because inner diaphragm 84 is permitted to roll upward, sensor chamber 94 does not experience a large volume decrease or a large corresponding pressure increase. Thus, greater force is applied to the anatomy of the patient through compressible ring 88 to neutralize tissue surrounding the artery without causing a corresponding large, error-producing change in pressure within sensor chamber 94 as the height of the side wall changes and the shape of flexible diaphragm 86 changes. As a result, the sensor 20 achieves more consistent and accurate blood pressure measurements.

Flexible diaphragm 86 is a generally circular sheet of flexible material capable of transmitting forces from an outer surface to fluid coupling medium 96 within sensor chamber 94. Diaphragm 86 is coupled to inner diaphragm 84 and is configured for being positioned over the anatomy of the patient above the underlying artery. Diaphragm 86 includes an active portion 98 and a nonactive portion 100 or skirt. Non-active portion 100 constitutes the area of diaphragm 86 where inner diaphragm 84 is heat sealed or bonded to diaphragm 86 adjacent compressible ring 88. Active portion 98 of flexible diaphragm 86 is not bonded to inner diaphragm 84, and is positioned below and within the inner diameter of ring 88. Active portion 98 of diaphragm 86 is the active area of sensing section 28 which receives and transmits pulse pressure to pressure transducer 90.

Fluid coupling medium 96 within sensor chamber 94 may consist of any fluid (gas or liquid) capable of transmitting pressure from flexible diaphragm 86 to transducer 90. Alternatively, another pressure pulse transmission medium may be used, including a medium made of a solid material or materials, or combinations of different materials, solid and fluid. Fluid coupling medium 96 interfaces between active portion 98 of diaphragm 86 and transducer 90 to transmit blood pressure pulses to transducer 90. Because fluid coupling medium 96 is contained within sensor chamber 94, which is isolated from compressible ring 88 of sensing section 28, fluid coupling medium 96 does not transmit blood pressure pulses parallel to the underlying artery, forces from the tissue surrounding the underlying artery, and other forces absorbed by compressible ring 88 to transducer 90. As a result, sensing section 28 more accurately measures and detects arterial blood pressure.

Sensing section 28 permits accurate and consistent calculation of blood pressure. Although blood pressure pulses are transmitted to the transducer 90 through hole 92, sensing section 28 is not dependent upon precisely accurate positioning of the sensor 20 over the underlying artery because of the large sensing surface of the active portion 98 of the flexible diaphragm 86. Thus, the sensor 20 is tolerant to patient movement as measurements are being taken.

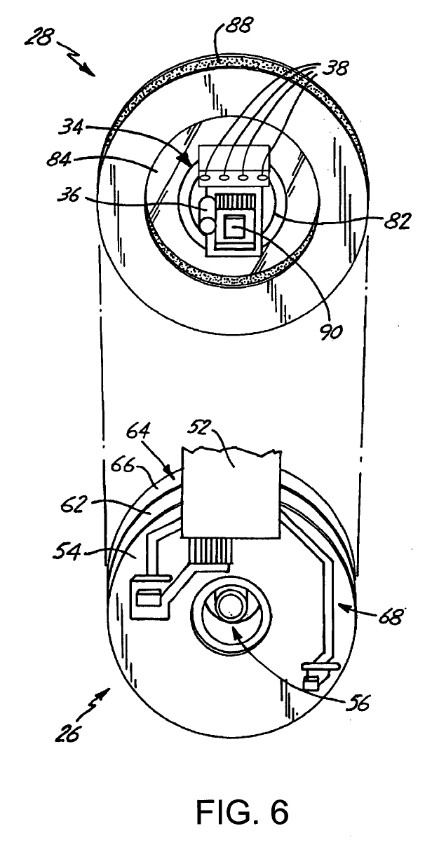

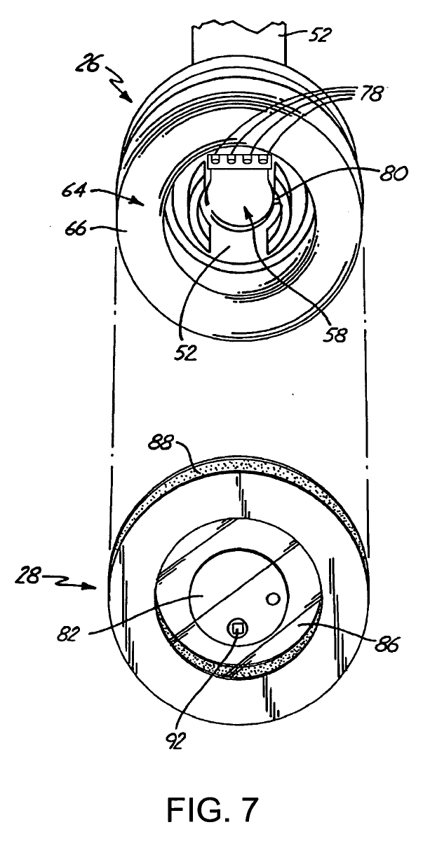

FIG. 6 is a top exploded view of base section 26 and sensing section 28 and FIG. 7 is a bottom exploded view of base section 26 and sensing section 28. Base section 26 includes electrical connector 52, top plate 54, upper receptacle 56, lower receptacle 58, inner mounting ring 60, outer mounting ring 62, flexible ring 64 (including side wall diaphragm 66), and electrical circuitry 68. Sensing section 28 includes diaphragm capture 82, inner diaphragm 84, flexible (or outer) diaphragm 86, compressible ring 88, pressure transducer 90 having sensing surface 92, and connection means 34. When assembled, flexible ring 64 and compressible ring 88 form the side wall of the sensor 20.

The connector 34 of sensing section 28 may be used to detachably connect sensing section 28 to base section 26, and also provides an electrical connection between the two units. The connector 34 extends from transducer 90 of sensing section 28 and is received by lower receptacle 58 of base section 26. The connector 34 may include an alignment element 36 and electrical connectors 38. Electrical connectors 38 are connected to and extend from pressure transducer 90. Electrical connectors 38 mate to corresponding electrical connectors 78 located within inner mounting ring 60 of lower receptacle 58. Electrical connectors 38 provide the connection between transducer 90 and electrical circuitry 68 of base section 26. Alignment element 36 is used to precisely position electrical connectors 38 with respect to the electrical connectors 78 of base section 26. Alignment element 36 of sensing section 28 is received by alignment receptacle 80 within inner mounting ring 60 of lower receptacle 58. Proper alignment between sensing section 28 and base section 26 is needed for the electrical connectors 38 to be connected to the electrical connectors 78. Sensing section 28 can be individually detached from base section 26 and replaced by another sensing section.

The sensor 20 achieves a zero pressure gradient across active portion 98 of the sensing section 28, achieves a zero pressure gradient between transducer 90 and the underlying artery, attenuates or dampens pressure pulses that are parallel to sensing surface 92 of transducer 90, and neutralizes forces of the tissue surrounding the underlying artery. The sensor 20 contacts and applies force to the anatomy of the patient across non-active portion 100 and active portion 98 of flexible diaphragm 86. However, the pressure within sensor chamber 94 is substantially equal to the pressure applied across active portion 98 of flexible diaphragm 86. In addition, because fluid coupling medium 96 within sensor chamber 94 is isolated from ring 88, pressure pulses parallel to the underlying artery, forces from tissue surrounding the underlying artery, and other forces absorbed by ring 88 are not transmitted through fluid coupling medium 96 to transducer 90. Consequently, the sensor 20 also achieves a zero pressure gradient between transducer 90 and the underlying artery. The remaining force applied by the sensor 20 across non-active portion 100, which neutralizes or offsets forces exerted by the tissue surrounding the underlying artery, is transferred through the side wall (rings 64 and 88) to top plate 54. As a result, the geometry and construction of the sensor 20 provides the proper ratio of pressures between non-active portion 100 and active portion 98 of flexible diaphragm 86 to neutralize tissue surrounding the underlying artery and to accurately measure the blood pressure of the artery.

If desired, sensing section 28 may be made detachably connected to base section 26 such that sensing section 28 may be replaced if contaminated or damaged. The blood pressure measurement device is typically used for non-invasively monitoring blood pressure in a hospital setting, by a physician or a patient. During use, the sensing section 28, which contacts the patient's anatomy, may become contaminated or damaged. In addition, the blood pressure measurement device may be used by multiple patients within one facility. To lower the costs associated with the blood pressure measurement device, it is desirable to have a low cost solution which enables the use of a single device with multiple patients. The present invention serves this purpose. To avoid contamination between patients and for more efficient use of the device by multiple patients, sensing section 28 is disposable and a new one is used for each patient. Sensing section 28, including pressure transducer 90, is detachable from base section 26. Sensing section 28 has a lower manufacturing cost than base section 26 because of the electrical circuitry associated with base section 26. A disposable sensing section 28 is desirable because it is less expensive to replace than an entire sensor interface assembly, including base section 26. Therefore, upon contamination or damage to the sensing section 28, the base section 26 may be retained while the sensing section 28 is disposed of and replaced.

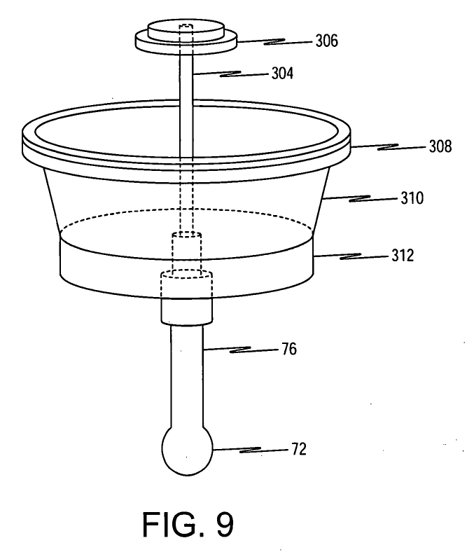

FIG. 8 is a cross-sectional view of the portable monitoring device 1, which contains a hold-down pressure generating unit 300 mounted to the casing 2. The hold-down pressure generating unit 300 includes a housing 302, a sensor post 76 terminating in the swivel ball 72, a piston 312, a diaphragm 310, a ring 308, a guide rod 304, and a guide rod mount 306. As shown in greater detail in FIG. 9, the diaphragm 310 preferably is a generally truncated conical molded rubber form, illustratively having a inner diameter at the large end of 1.37 inches, an inner diameter at the small end of 1.19 inches, and a height of 0.56 inches. Although shown in an extended position in FIG. 9, the diaphragm 310 preferably is made as a “rolling” diaphragm, which is to say, a diaphragm that is capable of assuming a collapsed condition as shown generally in FIG. 8. Preferably the diaphragm 310 is biased in the collapsed condition to help accelerate the release of hold-down pressure so that successive readings may be made more quickly. The diaphragm 310 is spaced from and cooperates with an interior air cavity formed within housing 302 to define a pressure chamber 301. The pressure chamber 301 extends generally above and partially around piston 312. The pressure chamber 301 receives pressurized air from a micro air pump 216, such that the diaphragm 310 expands and contracts to drive piston 312 and sensor post 76 up and down. As a result, a varying pressure may be applied to the piston 312 and the sensor post 76 so as to apply a desired hold-down pressure to the sensor 20, which is pivotally mounted to the lower end of the sensor post 76. By varying the volume of air within the pressure chamber 301, the device 1 applies a varying hold-down pressure to the patient's wrist and the underlying artery.

The diaphragm 310 is supported in place by ring 308. The ring 308 encircles the outer perimeter of the diaphragm 310 and captures an outer perimeter flange or edge portion of the diaphragm 310 between the ring 308 and the housing 302 so as to seal the diaphragm 310 against the housing 302. The ring 308 may be adhesively secured to the housing 302 and the diaphragm 310.

The piston 312 is preferably a disk or cylinder shaped member which has its top surface affixed to the diaphragm 310 in any desired manner, such as by an adhesive. A bore extends from top to bottom of the piston 312 and is sized for receiving a portion of the sensor post 76. The piston 312 mates with the sensor post 76 and exerts pressure upon the sensor post 76 and the sensor 20. As air is supplied to the pressure chamber 301, the volume of the pressure chamber 301 expands by moving the piston 312 downward. As air pressure is decreased by being vented through valve 206, the piston 312 lifts the sensor post 76 and thereby the sensor 20. The sensor post 76 terminates in the ball 72, which fits into a corresponding socket 74 in the sensor 20. As a result, the sensor 20 pivots when in contact with the wrist, so that the hold-down pressure generating unit 300 may operate automatically without the user having to adjust the sensor 20 to maintain it at the proper position on the wrist.

The guide rod 304 axially extends through a bore in the sensor post 76. The head of the guide rod 304 is secured to the housing 302 by the guide rod mount 306, which illustratively is a brass fitting having a hole which holds the guide rod 304 securely. The guide rod 304 guides the up and down movement of the piston 312 and the sensor post 76 as pressure within the pressure chamber 301 is varied. The guide rod 304 prevents lateral movement of the piston 312 and the sensor post 304 so that the sensor 20 apply perpendicular force to the patient's wrist. The guide rod 304 is held in place by the guide rod mount 306 while the piston 312 and the sensor post 76 move up and down.

The electrical systems of the monitoring device 1 include batteries 200, 202, and 204, user interface panel 220 which includes various displays and switches such as shown in FIG. 1, a printed circuit board 222 for the user interface panel 220, a controller printed circuit board 226 containing a microprocessor (not shown), and a printed circuit board spacer 224.

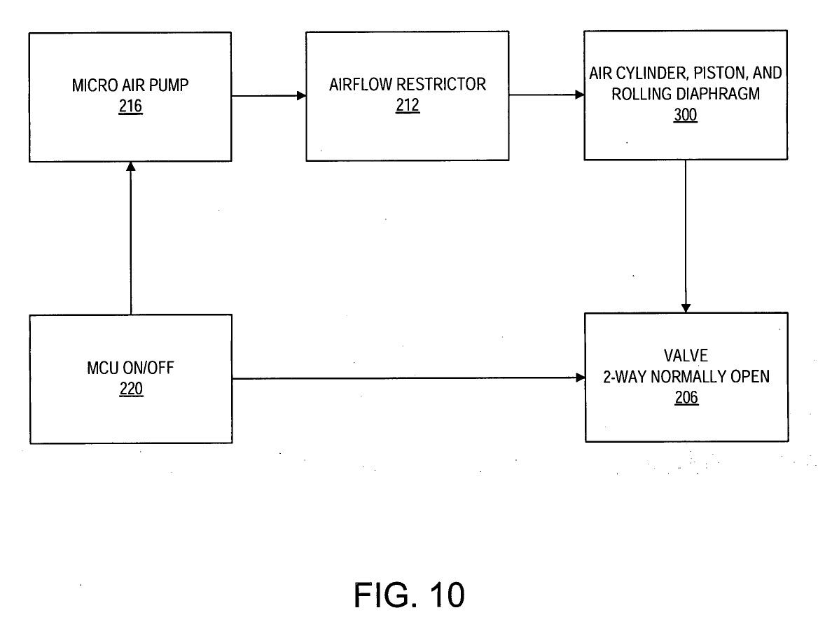

FIG. 10 is a block schematic representation of the pneumatic system shown in FIG. 8. When the start switch on the user interface panel 220 is pressed, the valve 206 closes. At the same time, the micro air pump 216 begins to pump air. The air passes from the pump 216 via tubing 214 through an airflow restrictor 212 to prevent an excessively rapid application of pressure to the patient. Air from the flow restrictor 212 further passes through tubing 210, 208 and 218 and into the air chamber 301 of the hold-down pressure generating unit 300 so as to extend the piston 312 and apply an increasing sweeping pressure to the patient's wrist via the sensor 20. When the pressurization phase is completed, power is removed and the valve 206 opens to rapidly vent the hold-down pressure from the air chamber 301 through tubes 218 and 208. Preferably the valve 206 is a normally open valve. A suitable valve 206 is an 111 series or 801 series miniature air valve such as type 1113085E which is available from Electrodyne, Inc. of Milwaukie, Oreg. A suitable micro air pump is model RWP08E01 which is available from Oken Ltd. of Tokyo, Japan. A suitable airflow restrictor may be made from a length of very flexible tubing having an inner diameter of 1/16 of an inch, in which a stainless steel set screw of type 3-48× 3/16″ is placed. The set screw has a diameter of 0.093 inches, which includes about 0.013 inches of thread. The air follows the thread path. Although the use of ambient air is particularly suitable because it avoids the need for a contained gas supply, a predetermined gas or predetermined mixture of gases may be used if desired.

The monitoring device 1 calculates blood pressure such as systolic blood pressure value and diastolic blood pressure value based upon the sensed pressure waveform data transmitted by transducer 90. The blood pressure values are calculated in any desired manner. One suitable manner calculates the blood pressure values with functions that use parameters derived from the waveform data and coefficients obtained from clinical tests upon patients having known blood pressure values. A suitable basic algorithm is described in U.S. Pat. No. 5,797,850 issued Aug. 25, 1998 to Archibald et al., which is incorporated herein in its entirety by reference thereto. Enhancements to the basic algorithm include a beat onset detection method as described in U.S. Pat. No. 5,720,292 issued Feb. 24, 1998 to Poliac, and a segmentation estimation method as described in U.S. Pat. No. 5,738,103 issued Apr. 14, 1998 to Poliac, which are incorporated herein in their entirety by reference thereto.

The base section 26 includes electrical circuitry 68 which transmits pressure data sensed by transducer 90 of sensing section 28 to a microprocessor (not shown) on the controller board 226. The sensed pressure data output of transducer 90 is typically an analog electrical signal representative of sensed pressure. The signal is amplified by an amplifier and applied to an input of an analog-to-digital converter. The A/D converter converts the analog signal to digital data which is transmitted to the electrical circuitry 68. Electrical circuitry 68 transmits the data to the microprocessor where a plurality of parameters are derived using the sensed pressure data received from transducer 90. The microprocessor determines a blood pressure value using the derived parameters, along with universal coefficients ascertained from clinical tests. The coefficients and the algorithm are stored in memory (not shown) on the controller board 226.

It will be appreciated that although the monitoring device 1 is described in the context of a wrist-mounted device, the monitoring device may be designed for use with other anatomical structures on which noninvasive monitoring of blood pressure may be performed, including the inside elbow, the ankle, and the top of the foot. Although the sensor 20 is described as having a distinct base section 26 and a distinct sensing section 28 which includes the pressure transducer 90, the sensor need not comprise distinct base and sensing sections. Although the sensor 20 is described as a unitary structure in which the pressure transducer 90 is mounted to the sensing section 28, various components of the sensor 20 such as the pressure transducer 90 may be distributed. As an example, the pressure transducer may be mounted to a different structure away from the base, and placed in fluid communication with the sensing surface through a fluid-filled tube.

It will therefore be appreciated that the description of the invention including its applications and advantages as set forth herein is illustrative and is not intended to limit the scope of the invention, which is set forth in the claims. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

1. An apparatus for monitoring a patient, comprising:
a body having a first attachment site spaced apart from a second attachment site over an intervening region of the body;
a pneumatically actuated pressure applicator mounted to the body;
a sensor comprising:
a support member movably coupled to the pressure applicator, and extendable and retractable relative to the intervening body region by the pressure applicator;
a pressure transducer; and
a pressure pulse transmission medium having a sensing surface for contacting tissue of the patient, the pressure transmission medium being supported by the support member and coupled to the pressure transducer for conveying pressure pulses thereto from the sensing surface;
a pneumatic pump mounted to the body and pneumatically coupled to the pressure applicator; and
a control system mounted to the body, the control system being electrically coupled to the pressure transducer and electrically coupled to the pump.

2. The apparatus of claim 1 further comprising a pressure release valve pneumatically coupled to the pressure applicator.

3. The apparatus of claim 2 wherein:
the pressure release valve is normally open; and
the control system is electrically coupled to the normally open pressure release valve for closing the valve during operation of the pneumatic pump.

4. The apparatus of claim 1 further comprising:
an airflow restrictor;
wherein the pneumatic pump is coupled to the pressure applicator through the airflow restrictor.

5. The apparatus of claim 1 wherein the pressure applicator comprises:
an air chamber; and
a diaphragm having an extended position and a retracted position, the diaphragm forming at least part of the air chamber, and the air chamber having an increased volume with the diaphragm in the extended position, and a decreased volume with the diaphragm in the retracted position;
the sensor being disposed generally away from the body with the diaphragm in the extended position, and disposed generally near to the body with the diaphragm in the retracted position.

6. The apparatus of claim 5 wherein the diaphragm is a rolling diaphragm biased toward the retracted position.

7. The apparatus of claim 1 further comprising:
a normally open pressure release valve pneumatically coupled to the pressure applicator;
wherein the control system is electrically coupled to the normally open pressure release valve for closing the valve during operation of the pneumatic pump; and
wherein the pressure applicator comprises:
an air chamber; and
a rolling diaphragm having an extended position and a retracted position and biased in the retracted position, the rolling diaphragm forming at least part of the air chamber, and the air chamber having an increased volume with the rolling diaphragm in the extended position, and a decreased volume with the rolling diaphragm in the retracted position;
the sensor being disposed generally away from the body with the rolling diaphragm in the extended position, and disposed generally near to the body with the rolling diaphragm in the retracted position.

8. The apparatus of claim 1 further comprising a wrist engaging member having a first end coupled to the first attachment site, and a second end coupled to the second attachment site.

9. The apparatus of claim 1 wherein the body comprises a fulcrum site, the apparatus further comprising:
a positioning guide coupled to the body in proximity to the fulcrum site;
wherein the positioning guide has an arc-like shape generally conformal with a cross-section of an anatomical structure from which blood pressure is detectable; and
wherein the positioning guide extends toward the sensor from the fulcrum site, the sensor passing through an opening in the positioning guide.

10. The apparatus of claim 9 further comprising:
an elongated sheet affixed to the body at the first attachment site and extending past the positioning guide in a direction generally tangential thereto;
wherein the positioning guide has a positioning notch therein for receiving a finger to detect a distal end of a radius bone when the positioning guide is engaged with a patient's wrist; and
wherein the elongated sheet has a hole therein for accessing the positioning notch with the finger.

11. The apparatus of claim 10 further comprising a wrist engaging member having a first end coupled to the first attachment site through the elongated sheet, and a second end coupled to the second attachment site.

12. The apparatus of claim 1 wherein the body comprises:
a first housing; and
a second housing rotatably coupled to the first housing about an axis transversely extending through the first housing and the second housing;
the pressure applicator being mounted in the second housing.

13. The apparatus of claim 12 wherein the second housing is swivelly connected to the first housing so that the second housing may be rotated about a plurality of axes with respect to the first housing.

14. The apparatus of claim 1 wherein the body is generally rigid.

15. The apparatus of claim 1 further comprising:
a flexible tube;
wherein the pressure pulse transmission medium comprises a fluid medium that fills the flexible tube; and
wherein the pressure transducer is mounted to the body, the flexible tube having one end coupled to the pressure transducer and another end in fluid communication with the sensing surface through the fluid medium.

16. The apparatus of claim 1 wherein:
the pressure pulse transmission medium comprises a fluid medium; and
the pressure transducer is mounted in proximity to the support member of the sensor and is in fluid communication with the sensing surface through the fluid medium.

17. The apparatus of claim 1 wherein the support member of the sensor is pivotally coupled to the pressure applicator.

18. The apparatus of claim 1 wherein the control system comprises:
a microprocessor;
a signal interface for coupling the microprocessor to the pressure transducer; and
a memory coupled to the microprocessor, the memory comprising computer-implementable instructions for calculating blood pressure from waveform signals from the pressure transducer and from pressure exerted by the pressure applicator.

19. The apparatus of claim 1 wherein the control system comprises:
a microprocessor; and
a memory coupled to the microprocessor, the memory comprising computer-implementable instructions for controlling the pump to generate a sweeping hold-down pressure.

20. The apparatus of claim 1 further comprising:
a positioning guide extending from the body toward the sensor, the positioning guide having a shape generally conformal with a cross-section of an anatomical structure from which blood pressure is detectable, and the sensor passing through an opening in the positioning guide; and
an elongated sheet extending from the first attachment site and past the positioning guide in a direction generally tangential thereto;
wherein the positioning guide has a positioning notch therein for receiving a finger to detect a distal end of a radius bone when the positioning guide is engaged with a patient's wrist; and
wherein the elongated sheet has a hole therein for accessing the positioning notch with the finger.

21. An apparatus for portably and non-invasively monitoring blood pressure, comprising:
a rigid casing having a fulcrum site;
a first anchor coupled to a first site on the casing;
a second anchor coupled to a second site on the casing, an intervening portion of the casing between the first and second sites forming a lever and the fulcrum site being on a first side of the lever;
a band having one end secured to the first anchor, and another end for being secured to the second anchor;
a housing contained within the casing, the housing forming a first part of an air chamber;
a rolling diaphragm having a truncated conical form when in an extend position, a large diameter end of the diaphragm being open and coupled to the housing, and a small diameter end of the diaphragm being closed for forming a second part of the air chamber, the rolling diaphragm being biased toward a collapsed position, and the air chamber having an increased volume with the rolling diaphragm in the extended position, and a decreased volume with the rolling diaphragm in the collapsed position;
a piston affixed to the small diameter end of the diaphragm;
a sensor post connected to the piston;
a guide rod connected to the housing, the sensor post being in slidable engagement with the guide rod;
a unitary pressure sensor comprising:
a sensor support member;
a flexible ring extending from the sensor support member;
a compressible ring extending from the flexible ring, a sensor interior being bounded by the sensor support member, the flexible ring, and the compressible ring;
a pressure pulse transmission medium contained generally within the sensor interior; and
a pressure transducer mounted within the sensor interior for receiving pressure pulses through the pressure pulse transmission medium;
the sensor support member having a sensor mount recessed within the flexible ring and pivotally connected to the sensor post, the sensor being disposed away from the casing with the rolling diaphragm in the extended position, and disposed near to the casing with the rolling diaphragm in the collapsed position;
a pneumatic pump contained within the casing;
an airflow restrictor contained within the casing, the pneumatic pump being pneumatically coupled to the airflow restrictor and the airflow restrictor being pneumatically coupled to the air chamber;
a normally open pressure release valve pneumatically coupled to the air chamber;
a control system contained within the casing and having a user interface accessible to a user from without the casing, the control system being electrically coupled to the pressure transducer, electrically coupled to the pump, and electrically coupled to the normally open pressure release valve for closing the valve during operation of the pneumatic pump;
a battery contained within the casing and electrically coupled to the control system; and
a positioning guide coupled to the casing in proximity to the fulcrum site, the positioning guide having an arc-like shape generally conformal with a cross-section of a human wrist; and extending toward the sensor from the fulcrum site;
wherein the positioning guide has a hole through which the sensor passes, and a positioning notch for receiving a finger to detect a distal end of a radius bone when the positioning guide in engaged with a patient's wrist; and
wherein the second anchor is elongated and extends from the casing and past the positioning guide in a direction generally tangential thereto, the second anchor having a hole therein for accessing the positioning notch with the finger.

22. An apparatus for portably and non-invasively monitoring blood pressure of a patient, comprising:
a body comprising a control system;
means for attaching the body to a monitoring site on an anatomical structure of the patient from which noninvasive monitoring of blood pressure may be performed;
means for pneumatically extending a sensor against the monitoring site from the body with a varying hold-down pressure, under control of the control system;
means for obtaining pressure data from the sensor, under control of the control system;
means for calculating arterial blood pressure from the pressure data, under control of the control system; and
means for pneumatically releasing the hold-down pressure from the sensor to retract the sensor, under control of the control system.

23. A method for portably and non-invasively monitoring blood pressure of a patient, comprising:
attaching a body to a monitoring site on an anatomical structure of the patient from which noninvasive monitoring of blood pressure may be performed;
pneumatically extending a sensor against the monitoring site from the body with a varying hold-down pressure, under control of a control system disposed in the body;
obtaining pressure data from the sensor, under control of the control system;
calculating arterial blood pressure from the pressure data, under control of the control system; and
pneumatically releasing the hold-down pressure from the sensor to retract the sensor.

24. The method of claim 23 wherein the releasing step is performed under control of the control system.

25. The method of claim 23 wherein the releasing step is performed automatically upon failure of the control system.
